(19) United States
(12) Patent Application Publication
(10) Pub. No.: US 2004/0140368 A1
(43) Pub. Date: Jul. 22, 2004

(54) Reusable air freshener

(76) Inventor: Anthony E. Girone, Toms River, NJ (US)

Correspondence Address: John O. Mingle, P.O. Box 1311, Laramie, WY, 82073-1311, US

(21) Appl. No.: 10/747,548

(22) Filed: Dec. 29, 2003

Related U.S. Application Data
(63) Continuation-in-part of application No. 10/350,571, filed on Jan. 24, 2003.
Application No. 10/350,571 is a continuation of application No. 10/228,327, filed on Aug. 26, 2002, now abandoned.
(60) Provisional application No. 60/314,550, filed on Aug. 24, 2001.

(51) Int. Cl. A61L 9/04

(52) U.S. Cl. 239/34

(57) ABSTRACT

An air freshener device designed to be efficient, simple and inexpensive that is utilized in many convenient ways and is formed in a wide variety of pleasant shapes and sizes, while at the same time being reusable with different scents by just washing and recharging.

This application is a continuation-in-part of application Ser. No. 10/350,571 filed Jan. 24, 2003, now abandoned, which itself was a continuation of application Ser. No. 10/228,327, filed Aug. 26, 2002, now abandoned, which itself was a continuation of Provisional Application Serial No. 60/314,550, filed Aug. 24, 2001.

### BACKGROUND OF INVENTION

1. Field of Invention.

The present invention relates to an apparatus used as a practical air freshener and the manner in which it can be constructed with emphasis on reuse.

2. Background.

Air fresheners are a common item and have been with our society for scores of years. Many designs and types exist and they have in common a method of passing air over a substance that is giving off a specific air freshener scented vapor. These air fresheners are made in various shapes and sizes and positioned in places around the home, garage and car.

Many of these air fresheners have elaborate procedures to pass enough air over the freshening substance to produce the desired effect. Conversely the subject invention primarily employs material that air passes through readily by natural means. Generally these air fresheners are not reusable and are thrown away after their ability to continue to be an adequate air freshener expires. Conversely the subject invention is reusable either by recharging with the same freshening substance, or if a different scent is utilized, just wash before the new application.

Related United States patents include:

| No. | Inventor | Year |
|---|---|---|
| 6,254,836 | Fry | 2001 |
| 6,089,947 | Green | 2000 |
| 6,048,248 | Ritchey | 2000 |
| 5,788,061 | Hammond | 1998 |
| 5,823,432 | Hogan | 1998 |
| 5,503,332 | Glenn | 1996 |
| 5,383,598 | Styles | 1995 |
| 4,465,232 | Field | 1984 |
| 1,960,149 | Gilot | 1934 |

Fry discloses an air freshener that employs an internal bulb to force air over liquid scented oil before it leaves through selected apertures. This special dispensing apparatus is situated within a much larger body that is identified with artistic shapes.

Green discloses an air freshener in the form of an animal having a hollow body and mouth Scented pellets are entered into the body through the mouth and released through air-permeable material forming certain aspects of the body.

Ritchey discloses a specially sewed, elongated fabric shell that holds stuffing material held in place with two sets of special flaps. The stuffing can contain scented material, such as catnip.

Hammond discloses a two compartment potpourri container in the shape of an envelope that can be either be flat for shipping or popped up to an A-frame configuration for use and composed of paper or breathable plastic.

Hogan discloses an air freshener device as a form that assumes the shape of an article of clothing into which it is inserted. The form holds an air permeable pouch to receive scented beads.

Glenn discloses a scent packet composed of a wick sheet containing perfume oil that is wrapped by a plastic film, and then placed with a plastic bag, which itself is inserted into a paper envelope. The inner layers are designed to slow up the release of the scent thus producing a long lasting air freshener.

Styles discloses an air freshener retainer made of matched opposing sides sewn together to form a pocket for scented material, and further composed of material not allowing the scented material to contaminate contacted surfaces. Separate apertures are provided for needed air circulation.

Field discloses an adornment designed to be connected to clothing. The stitched fabric forms a pocket to place a scented material.

Gilot discloses foldable paper container surrounding a bag holding scented material and formed so that by unfolding the container air aperture holes are exposed allowing the vapor to escape.

### SUMMARY OF INVENTION

The objectives of the present invention include overcoming the above-mentioned deficiencies in the prior art by making an air freshener device that is convenient, pleasant and reusable while being inexpensive.

### BRIEF DESCRIPTION OF DRAWING

FIG. 1 shows a view of a reusable air freshener in the shape of a typical face.

### DETAILED DESCRIPTION OF INVENTION

The subject invention is a simple air freshener made in many pleasant shapes allowing it to be utilized in any convenient manner while at the same time being reusable with different scents by just washing and recharging.

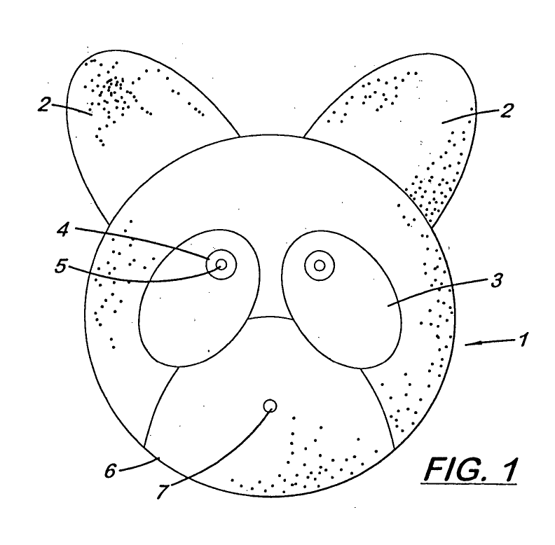

FIG. 1 shows a typical configuration of the subject invention using pieces where a carpet-like piece for the back material 1 of circular shape is employed to form a basic face by adding ears 2 fixed in a similar manner from partial circles. The face is then composed of various circles and ellipses of air-passing material for the inner eyes 4 and outer eyes 5 positioned on top of cheeks 3 with the mouth 7 and chin 6 all composed similarly by fastening onto the back side 1. This composite now forms at least one inner pocket that can hold a scented substance. For convenience as well as support the back material 1 is often doubled or folded over. Although this is in the form of a face, many other pleasant shapes are possible in the best mode formed from geometric basics.

This design is all washable so that if the scent is to be changed, the air freshener is just washed before recharging with a different scented substance.

The simplicity of such design keeps the cost low and at the same time provides a wide variety of pleasant, recognizable shapes available for staging such air fresheners. The shape of the air freshener shown in FIG. 1 is for explanatory purposes only and does not restrict the shape of additional air fresheners produced under the subject invention.

An apparatus for an air freshener comprising a backside material that is structurally supporting and specifically shaped and an array of front-side material that allows vapor penetration. The front side material fastens onto said backside material in a manner to create a recognizable shape and make at least one inner pocket to receive a perfumed substance. The perfumed substance further comprises a synthetic chemically treated and aromatically emissive perfume or a naturally occurring perfume. The front-side material is commonly fake-fur or felt or both, although other material allowing vapor penetration is usable. The backside material is commonly carpet since it is represents a good supporting material; however, often this carpet is doubled for convenience. All the material is washable allowing the perfumed substance to be easily changed by washing and inserting a different odor-producing substance, and thus the air freshener is reusable. The front-side material fastens onto said backside material in the best mode with the use of glue; yet a myriad of other common fasteners are possible. Velcro is also a potential fastener and further allows the user to modify the recognizable shape formed by the front-side material. The inner pocket or pockets are commonly formed using a slit opening in the backside material; yet, if the array of front-side material is left unfastened along an edge, this also creates such a perfume pocket.

An apparatus for an air freshener comprising a front-side material and a carpet backside material, both washable, wherein the front-side material allowing air ventilation is composed of one or more individual pieces of fake-fur and felt that compose a recognizable shape, and further wherein the individual pieces are glued onto the backside material forming at least one inner pocket to receive a perfumed substance.

The foregoing description of the specific embodiments will so fully reveal the general nature of the invention that others can, by applying current knowledge, readily modify and/or adapt for various applications such specific embodiments without departing from generic concept, and therefore such adaptations or modifications are intended to be comprehended within the meaning and range of equivalents of the disclosed embodiments. It is to be understood that the phraseology or terminology herein is for the purpose of description and not of limitation.

What is claimed is:

1. An apparatus for an air freshener comprising:
a washable backside material that is structurally supporting and specifically shaped;
an array of washable front-side material that allows vapor penetration;
fasten said front-side material onto said backside material in a manner to create a recognizable shape; and
make at least one inner pocket to receive a perfumed substance.

2. The apparatus according to claim 1 wherein said permitted substance further comprises a synthetic chemically treated and aromatically emissive perfume.

3. The apparatus according to claim 1 wherein said perfumed substance further comprises a naturally occurring perfume.

4. The apparatus according to claim 1 wherein said front-side material further comprises fake-fur.

5. The apparatus according to claim 1 wherein said front-side material further comprises felt.

6. The apparatus according to claim 1 wherein said backside material further comprises carpet.

7. The apparatus according to claim 1 wherein said fasten said front-side material onto said backside material further comprises the use of glue.

8. The apparatus according to claim 1 wherein said fasten said front-side material onto said backside material further comprises the use of Velcro.

9. The apparatus according to claim 1 wherein said make at least one inner pocket further comprises a slit opening in said backside material.

10. The apparatus according to claim 1 wherein said make at least one inner pocket further comprises said array of front-side material unfastened along an edge.

11. An apparatus for an air freshener comprising:
a front-side material and a carpet backside material, both washable, wherein said front-side material allowing air ventilation is composed of one or more individual pieces of fake-fur and felt that compose a recognizable shape, and wherein said individual pieces are glued onto said backside material forming at least one inner pocket to receive a perfumed substance.
